(19) United States
(12) Patent Application Publication
(10) Pub. No.: US 2016/0209410 A1
(43) Pub. Date: Jul. 21, 2016

(54) TEST PIECE FOR IMMUNOCHROMATOGRAPHY, DEVELOPING FLUID USED THEREFOR, AND IMMUNOCHROMATOGRAPHY USING THE SAME

(71) Applicant: FURUKAWA ELECTRIC CO., LTD., Tokyo (JP)

(72) Inventors: Masayuki FUKUSHIMA, Tokyo (JP); Michio OHKUBO, Tokyo (JP); Kazutomi MIYOSHI, Tokyo (JP); Takanari SEKINE, Tokyo (JP)

(73) Assignee: FURUKAWA ELECTRIC CO., LTD., Tokyo (JP)

(21) Appl. No.: 14/988,012

(22) Filed: Jan. 5, 2016

Related U.S. Application Data
(63) Continuation of application No. PCT/JP2014/069671, filed on Jul. 25, 2014.

(30) Foreign Application Priority Data
Jul. 25, 2013 (JP) 2013-154763

(51) Int. Cl. G01N 33/543

(52) U.S. Cl. CPC G01N 33/54386

(57) ABSTRACT

A test piece for immunochromatography, containing an aggregation inhibiting pad, a conjugate pad, and a membrane, in which the membrane contains a test area for capturing a target substance, the aggregation inhibiting pad contains a desalting agent, and the conjugate pad contains a labeling agent.

### CROSS-REFERENCE TO RELATED APPLICATIONS

This application is a Continuation of PCT International Application No. PCT/JP2014/069671 filed on Jul. 25, 2014, which claims a priority under 35 U.S.C. §119 (a) to Japanese Patent Application No. 2013-154763 filed in Japan on Jul. 25, 2013. Each of the above applications is hereby expressly incorporated by reference, in its entirety, into the present application.

### TECHNICAL FIELD

The present invention relates to a test piece for immunochromatography, developing fluid used therefor, and immunochromatography using the same.

### BACKGROUND ART

As a method of detecting a trace amount substance contained in a living body, e.g., an antibody, there is a medical test based on lateral flow type immunochromatography. According to the test, an analyte substance contained in an analyte is captured by labeling particles, and then the particles are transported along a porous support based on capillary phenomenon. Then, the analyte substance is brought into contact with a capturing substance which is immobilized in the porous support. As a result, the analyte substance is captured and concentrated, and a target substance exhibits color at a part immobilized with the capturing substance. Based on the color exhibition, it can be determined as to the presence or the absence of the analyte substance. Regarding such an immunochromatography, three features can be pointed as follows:

(1) Prompt test can be made, as the time is short to reach the identification.
(2) Simple operation, as the test can be made only by applying an analyte.
(3) Easy determination, as no special detection device is required.

By taking advantage of those characteristic aspects, immunochromatography is used for a pregnancy test reagent or for an influenza test reagent, and also utilized as a new method of POCT (Point of Care Testing). Further, for example, in a field of food testing, it receives more attention and is popularized as a reagent for detecting a food allergen.

In view of the features about the immunochromatography described above, the applicant studied for developing a reagent for application to a membrane. As a result, the applicant developed a technique of using fluorescent silica fine particles (see Patent Literature 1, etc.). As a result, compared to conventional technologies in which a biological substance or cells from a living body, or colloidal gold particle is used, measurement and detection can be attained at low cost with significantly high stability. In addition, as a need like improving detection sensitivity and quantification can be met more appropriately, it makes a contribution to broadening the application area of immunochromatography.

### CITATION LIST

### Patent Literatures

Patent Literature 1: WO 2008/018566 pamphlet

Patent Literature 2: Japanese Patent No. 4578570

### SUMMARY OF INVENTION

### Technical Problem

When the present inventors further continued research on an immunochromatography technology using the above-described fluorescent fine particles, it has been found that fine particles forming a labeling agent aggregate to cause difficulty in migration within a test piece in several cases. Specifically, the following phenomenon was observed. An analyte is often subjected to sterilization treatment with an alkali or acid upon being applied to immunochromatography. The analyte after the treatment as it is damages a member such as a membrane, and therefore ordinarily requires neutralization treatment before a test. Under an influence by salt produced in this neutralization, the labeling agents aggregate within the membrane, resulting in stagnation without migrating to a test area in several cases. As a result, labeling particles that have captured a target substance do not arrive at the test area to cause difficulty in detection with high sensitivity (see Patent Literature 2 described above for an example in which no alkali treatment is applied, and an analyte is diluted with a tris-hydrochloric acid buffer solution, and EDTA is applied thereto). This influence becomes significant particularly in a labeling agent using silica fine particles, and an improvement thereof was required.

The present invention is contemplated for providing a test piece for immunochromatography in which good flowability of the labeling agent can be secured even upon applying sterilization treatment by an alkali or acid to the analyte, and a target substance can be detected or measured with higher sensitivity and accuracy. Further, the present invention is contemplated for providing developing fluid used therefor and immunochromatography using the same.

### Solution to Problem

The problems are solved by the following means.

[1] A test piece for immunochromatography, comprising:

an aggregation inhibiting pad;

a conjugate pad; and

a membrane;

wherein the membrane contains a test area for capturing a target substance;
wherein the aggregation inhibiting pad contains a desalting agent; and
wherein the conjugate pad contains a labeling agent.
[2] The test piece for immunochromatography described in the above item [1], wherein the desalting agent is a chelating agent or an aptamer.
[3] The test piece for immunochromatography described in the above item [2], wherein the chelating agent is an aminocarboxylic acid-series chelating agent.
[4] The test piece for immunochromatography described in any one of the above items [1] to [3], wherein the labeling agent is fluorescent silica fine particles.
[5] The test piece for immunochromatography described in any one of the above items [1] to [4], wherein a connecting molecule is introduced into the aggregation inhibiting pad, and the desalting agent is introduced thereinto through the connecting molecule.
[6] Developing fluid for immunochromatography, comprising:

an alkali or acid;

a target substance; and

a desalting agent.

[7] The developing fluid for immunochromatography described in the above item [6], wherein the desalting agent is a chelating agent or an aptamer.
[8] The developing fluid for immunochromatography described in the above item [7], wherein the chelating agent is an aminocarboxylic acid-series chelating agent.
[9] The developing fluid for immunochromatography described in any one of the above items [6] to [8], which is treated through a step of providing analyte liquid with an alkali or acid, a step of heating, and a step of neutralization with the alkali or acid.
[10] Immunochromatography, which is performed using the test piece described in any one of the above items [1] to [5], wherein the test piece is provided with developing fluid containing an alkali or acid and a target substance, to detect a target substance in the analyte liquid.
[11] Immunochromatography, wherein a test piece for immunochromatography provided with a membrane having a test area for capturing a target substance is provided with the developing fluid containing a desalting agent described in any one of the above items [6] to [9], to detect the target substance.
[12] Immunochromatography for performing testing by providing developing fluid containing an alkali or acid and a target substance and allowing passage through a membrane to capture the target substance in a test area of the membrane,
wherein the developing fluid is desalted by using a desalting pad into which a desalting agent is incorporated, or applying desalting treatment to analyte liquid, to prevent aggregation of the target substance.

### Advantageous Effects of Invention

According to the test piece for immunochromatography, developing fluid used therefor and immunochromatography using the same of the present invention, good flowability of a labeling agent can be secured, even upon applying sterilization treatment by an alkali or acid to an analyte, and a target substance can be detected or measured with higher sensitivity and accuracy. In particular, a significant effect is exhibited in a labeling agent using silica fine particles to preferably migrate the agent within of a membrane, and to achieve satisfactory detection of the target substance, or the like.

Other and further features and advantages of the invention will appear more fully from the following description, appropriately referring to the accompanying drawings.

### BRIEF DESCRIPTION OF DRAWINGS

FIG. 1 is an exploded perspective view schematically illustrating a long test stick that can be preferably used in the present invention.

FIGS. 2(a) and 2(b) are diagrams illustrating a test strip that can be preferably used in the present invention, in which FIG. 2(a) is a plan view and FIG. 2(b) is an expanded cross-sectional view.

FIG. 3 is a partial expanded cross-sectional view schematically illustrating a modified example of a test strip that can be preferably used in the present invention.

FIG. 4 is a partial expanded cross-sectional view schematically illustrating another modified example of a test strip that can be preferably used in the present invention.

FIG. 5 is graphs illustrating results (fluorescence intensity) of tests of detecting a target substance carried out in Example and Comparative Example.

FIG. 6 is a graph illustrating a migration state (particle accumulation ratio) of labeling particles within a membrane.

FIG. 7 is a photograph substituted for drawing illustrating an electron micrograph obtained by observing an inside of a membrane after a test is carried out in Comparative Example.

FIGS. 8(a) and 8(b) are graphs illustrating a particle size distribution of silica particles obtained in Reference Example in dispersion liquid.

FIG. 9 is a micrograph illustrating results (state of migration of labeling particles within a membrane) of an experiment using an aggregation pad used in Example.

FIG. 10 is another micrograph illustrating results (state of migration of labeling particles within a membrane) of an experiment using an aggregation pad used in Example.

### DESCRIPTION OF EMBODIMENTS

A preferred embodiment of a test piece for immunochromatography according to the present invention is described, referring to drawings.

### [Test Strip (Test Piece)]

In the test strip (test piece) for immunochromatography that can be used in the present embodiment, the following members are preferably connected to each other in series so as to cause a capillary phenomenon.

Aggregation inhibiting pad 8a

Conjugate pad (member obtained by impregnation with labeling unit and drying it) 8b

Membrane (antibody-immobilized membrane) 8c

Absorbent pad 8d

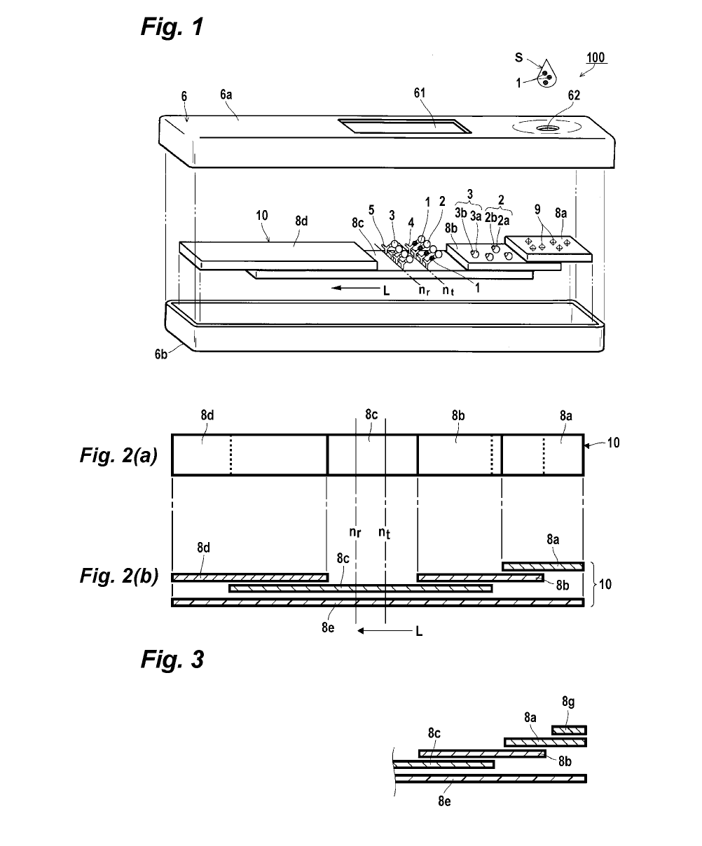

According to this embodiment, a test strip 10 provided with a membrane 8c having the above described test area nₜ and the reference area nᵣ is sandwiched between an upper casing part 6a and a lower casing part 6b to form a long test body 100 as illustrated in FIG. 1 and FIGS. 2(a) and 2(b). On the upper casing part 6a, a detection opening part 61 and an opening part 62 for introducing an analyte are provided. Through the detection opening part 61, irradiation light is supplied to the test strip 10 present inside, and the fluorescence and light absorption emitted therefrom can be detected and measured. Meanwhile, by supplying analyte liquid S to the test strip 10 through the opening part 62 for introducing an analyte, measurement test can be carried out.

One preferred embodiment of the test strip for immunochromatography according to the present embodiment is described, referring to FIGS. 2(a) and 2(b). However, the present invention is not restricted by this.

FIG. 2(a) shows a plan view of one preferred embodiment of a test strip for immunochromatography according to the present invention, and FIG. 2(b) is a diagram illustrating an expanded longitudinal cross-sectional view of the test strip for immunochromatography shown in FIG. 2(a). A test strip 10 for immunochromatography of the present embodiment is provided with an aggregation inhibiting pad 8a, a conjugate pad 8b, an antibody-immobilized membrane 8c and an absorption pad 8d, as mentioned above. Further, each of the above-described constitutional member is preferably lined with a backing sheet 8e added with an adhesive, as in the present embodiment.

### (Target Substance)

In the present invention, the target substance 1 of the subject of detection and quantification includes antigens, antibodies, DNAs, RNAs, sugars, polysaccharides, ligands, receptors, peptides, chemical substances and the like. In the present invention, a sample containing the target substance 1 is no particularly limited, but examples of the sample includes liquid samples such as urine and blood.

### (Aggregation Inhibiting Pad)

In the present embodiment, the aggregation inhibiting pad 8a is a constitutional member onto which a sample (analyte) containing a target substance is added dropwise. A material, size or the like of the aggregation inhibiting pad is not particularly limited, and a general one applied to the sample pad can be used, for example. This aggregation inhibiting pad 8a is provided with a desalting agent 9 described later.

A method of preparing the aggregation inhibiting pad is not particularly limited. Specific examples include a method of impregnating a desalting agent into a membrane material used for the sample pad or conjugate pad. Specifically, the aggregation inhibiting pad can be obtained by immersing the membrane material into solution of the desalting agent, and then drying the resultant material. Alternatively, specific examples include a method of applying, adding dropwise or spraying solution or dispersion liquid of the desalting agent onto the membrane material, and then drying the resultant material. An application amount of the desalting agent is not particularly limited, but the content of the desalting agent per unit area (cm²) in the pad 8a is preferably 1 μg to 1,000 μg.

In addition thereto, specific examples of a method of immobilizing the desalting agent onto the pad material include immobilization by freeze drying. Specifically, 1% EDTA solution is impregnated into a conjugate pad made of glass fiber, and then frozen at −80° C. to −10° C. After freezing, freeze drying treatment is applied thereto to immobilize the resultant material onto the pad.

Moreover, specific examples of another method include a method of introducing a connecting molecule into the above-described aggregation inhibiting pad to introduce the desalting agent (preferably having a connecting moiety) through the connecting molecule. Connection between the pad and the connecting molecule or between the connecting molecule and the desalting agent is preferably made by a covalent bond between compounds. For example, an SH group is added to the glass fiber by using a silane coupling agent (connecting molecule) such as MPMS (3-methacryloxypropyltrimethoxysilane) having the SH group, and then a chelate compound (desalting agent having the connecting moiety) having a maleimide structure as shown in the following scheme can be covalently bonded thereto. An order of introduction is not particularly limited. The connecting molecule may be introduced into the pad, and then the desalting agent may be introduced thereinto. Alternatively, the connecting molecule and the desalting agent may be connected in advance, and then introduced into the pad. When a combination having binding properties is applied, the desalting agent may be directly connected (covalent bond or the like) to the pad without using any connecting molecule. The desalting pad immobilized with the desalting agent (aggregation inhibitor) is preferably arranged on a side upstream of the membrane in an immunochromatography kit.

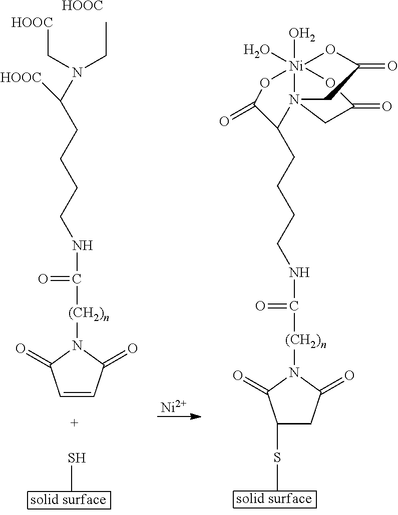

### (Sample Pad)

Although no sample pad is used in the test strip in the form shown in FIG. 1, a sample pad 8g may be provided in an upper part of the aggregation inhibiting pad, as in the modified example shown in FIG. 3. The sample pad 8g is a constitutional member to which a sample containing a target substance is loaded. The material and the size of the sample pad are not specifically limited. Those commonly used for a product of the same type can be used.

### (Conjugate Pad)

The conjugate pad 8b is a constitutional member impregnated with labeling reagent silica fine particles (labeling unit) 2 and 3, in which a target substance included in a sample, which migrates from the aggregation inhibiting pad 8a based on capillary phenomenon, is captured and labeled by the labeling reagent silica fine particles (labeling unit) as a result of a specific molecular recognition reaction such as antigen and antibody reaction.

The content of the labeling reagent silica fine particles (labeling unit) per unit area (cm²) of the conjugate pad 8b is, although not particularly limited, preferably from 1 μg to 100 μg. Examples of the impregnation method include a method of coating, applying or spraying dispersion of the labeling reagent silica fine particles, and then drying.

### (Antibody-Immobilized Membrane)

In an antibody immobilizing part in the antibody-immobilized membrane 8c, the test line nₜ immobilized with an antibody for capturing a target substance is provided to determine presence of the target substance, i.e., to determine a positive response or a negative response. The antibody-immobilized membrane 8c has preferably a control line nᵣ immobilized with an antibody for capturing the labeling reagent silica fine particles.

The membrane 8c is a constitutional member in which the target substance 1 labelled by the silica fine particles (labeling unit) 2 and 3 migrates based on capillary phenomenon, and has an antibody immobilizing part (determination part) which causes a reaction to form a sandwich-type immunocomplex composed of immobilized antibody-target substance-labeling reagent silica fine particles. The shape of the antibody immobilizing part (determination part) in the membrane is not particularly limited as long as a capturing antibody is locally immobilized, and examples thereof include a line shape, a circular shape, a band shape, or the like. Among these, the line shape is preferable, and the line shape with width of from 0.5 to 1.5 mm is more preferable.

Although the antibody immobilization amount in each of the antibody immobilizing part (test area) nₜ is not particularly limited, when it has a line shape, it is preferably from 0.5 μg to 5 μg per unit length (cm). Examples of the immobilization method include a method of coating, applying or spraying antibody solution, drying it, and immobilizing the antibody by physical adsorption. To avoid an influence of non-specific adsorption on measurement after antibody immobilization described above, the entire antibody-immobilized membrane is preferably subjected to so-called blocking treatment in advance. For example, a method of impregnating in buffer solution containing a blocking agent such as albumin, casein, and polyvinyl alcohol for an appropriate time followed by drying can be mentioned. Examples of a commercially available blocking agent include skim milk (manufactured by DIFCO) and 4% Block Ace (manufactured by Meiji Dairies Corporation).

The membrane 8c also contains the reference area nᵣ, in which the labeling particles (labeling unit) 3 which cannot capture the target substance are captured. Accordingly, presence or absence, or amount against the target substance can be determined compared to fluorescence/light absorption from the test area nₜ. To perform this function, the test purpose labeling unit 2 consists of the silica fine particles 2a and the test purpose binding substance 2b. The test purpose binding substance 2b has the ability to bind to the target substance. Meanwhile, the reference purpose labeling unit 3 consists of the silica fine particles 3a and the reference purpose binding substance 3b. The reference purpose binding substance does not have the ability to bind to the target substance, but has the ability to bind to the reference purpose capturing substance.

### (Absorption Pad)

The absorption pad 8d is a constitutional member for absorbing an analyte S (target substances) which migrates along the membrane based on capillary phenomenon and also the labeling reagent silica fine particles (labeling units) 2 and 3, and generating a constant flow of them at all times.

The material of each of the aforementioned constitutional members is not particularly limited. Instead, members used for a test strip for immunochromatography can be used. Preferred examples of the sample pad and the conjugate pad include a pad of glass fiber such as Glass Fiber Conjugate Pad (trade name, manufactured by MILLIPORE). Preferred examples of the membrane include a nitrocellulose membrane such as Hi-Flow Plus120 (trade name, manufactured by MILLIPORE). Preferred examples of the absorption pad include a cellulose membrane such as Cellulose Fiber Sample Pad (trade name, manufactured by MILLIPORE).

Examples of the backing sheet added with adhesives include AR9020 (trade name, manufactured by Adhesives Research).

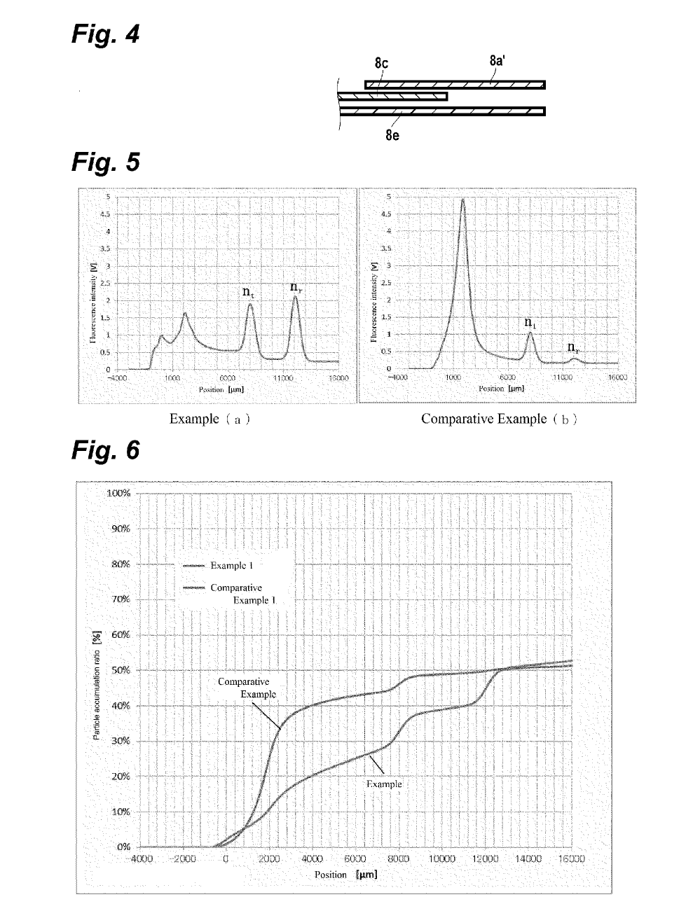

FIG. 3 and FIG. 4 are expanded cross-sectional views illustrating modified examples of test pieces for immunochromatography as related to a preferred embodiment according to the present invention. In the example in FIG. 3, a sample pad 8g is applied in an upper part of an aggregation inhibiting pad 8a. Thus, a material of the sample pad is optimized to allow promotion of further ensured absorption and migration to the inside of the analyte. In the example in FIG. 4, an aggregation inhibiting pad 8a′ is impregnated with a labeling unit, and serves also as a function of the conjugate pad. Thus, the number of members can be decreased and a simpler and less expensive test piece can be provided. Also in these modified examples, the desalting agent impregnated into the aggregation inhibiting pad migrates with mobilization of the analyte, and is brought into contact with the labeling agent (silica fine particles or the like) to exhibit a good aggregation inhibiting effect.

### [Desalting Agent]

Although a kind of desalting agent is not particularly limited in the present embodiment, the desalting agent is preferably a compound that is added in acid or base neutralization treatment after analyte treatment to remove a salt component that causes aggregation of the labeling agents. Examples of the salt component include an alkali component, and specific examples include an alkali metal, an ion thereof or a salt thereof (for example, sodium, potassium or lithium, an ion thereof or a salt thereof, or the like), and an alkaline earth metal, an ion thereof, or a salt thereof (for example, calcium, magnesium or barium, an ion thereof or a salt thereof, or the like). Alternatively, examples of the salt component include an acid component, and specific examples include hydrochloric acid, an ion thereof or a salt thereof, hydrofluoric acid, an ion thereof or a salt thereof, or the like. Further, the desalting agent is preferably a compound having adsorbability to the above-described salt component, and preferably a ligand compound (chelating agent) that forms a metal complex with these metals. Specific examples of the chelating agent include an organic compound having a hetero atom, and more specific examples include a nitrogen-containing hydrocarbon compound, an oxygen-containing hydrocarbon compound and a sulfur-containing hydrocarbon compound.

The nitrogen-containing hydrocarbon compound is preferably a compound having an amino group (NR_{N}²) or an imino group (NR_{N}) in its molecule. The compound is further preferably a compound having a carboxyl group together with the amino group (NR_{N}²) or imino group (NR_{N}). On this occasion, the compound may have an ether group (O) in its structure thereof. Here, R_{N} is a hydrogen atom, an alkyl group having 1 to 12 carbon atoms, an alkenyl group having 2 to 12 carbon atoms or an aryl group having 6 to 14 carbon atoms. The alkyl group, the alkenyl group or the aryl group may further have a substituent, and specific examples of the substituent include a hydroxy group or a carboxyl group.

Further specific examples include a compound having a structure of >N—(CH₂)ₙ—COOH (n is an integer from 1 to 4) or a structure of —N—(CH₂)ₙ—COOH)₂ in its molecule.

The molecular weight of the compound forming the desalting agent is not particularly limited. Taking an example of this kind of typical chelating agent into consideration, the molecular weight is preferably about 50 to about 1,000.

The above-described chelating agent is preferably an aminocarboxylic acid-based chelating agent (preferably having 2 to 24 carbon atoms, more preferably having 2 to 12 carbon atoms and particularly preferably having 2 to 10 carbon atoms). Specific examples include the following compounds. The number of carboxyl groups in the aminocarboxylic acid-based chelating agent is preferably 1 to 12, more preferably 1 to 8, and particularly preferably 1 to 6. The number of amino groups or imino groups in the aminocarboxylic acid-based chelating agent is preferably 1 to 8, more preferably 1 to 6, and particularly preferably 1 to 4.

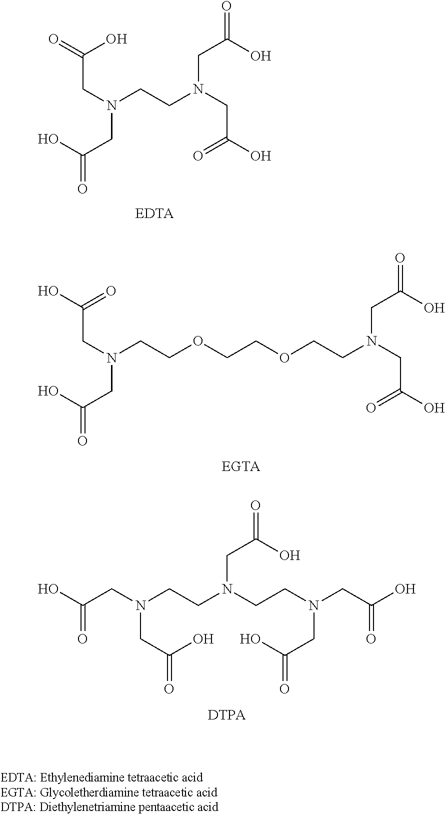 [SMILES: O=C(O)CN(CCN(CC(=O)O)CC(=O)O)CC(=O)O ; O=C(O)CN(CCOCCOCCN(CC(=O)O)CC(=O)O)CC(=O)O ; O=C(O)CN(CCN(CC(=O)O)CC(=O)O)CCN(CC(=O)O)CC(=O)O]

In addition thereto, specific examples of the nitrogen-containing hydrocarbon compound as the chelating agent include ethylenediamine, a compound having an imidazole moiety, a compound having a pyrazole moiety, a compound having a triazole moiety, a compound having a piperazine moiety, a compound having a piperidine moiety, a compound having a morpholine moiety, a compound having a pyrrolidine moiety, a compound having a thiazole moiety and a compound having a pyrrole moiety. Further, specific examples of the oxygen-containing hydrocarbon compound include a compound having a furan moiety, an ether compound (crown ether: C8 to C24) and a carboxylic acid compound (e.g. citric acid, succinic acid, phthalic acid, maleic acid or the like).

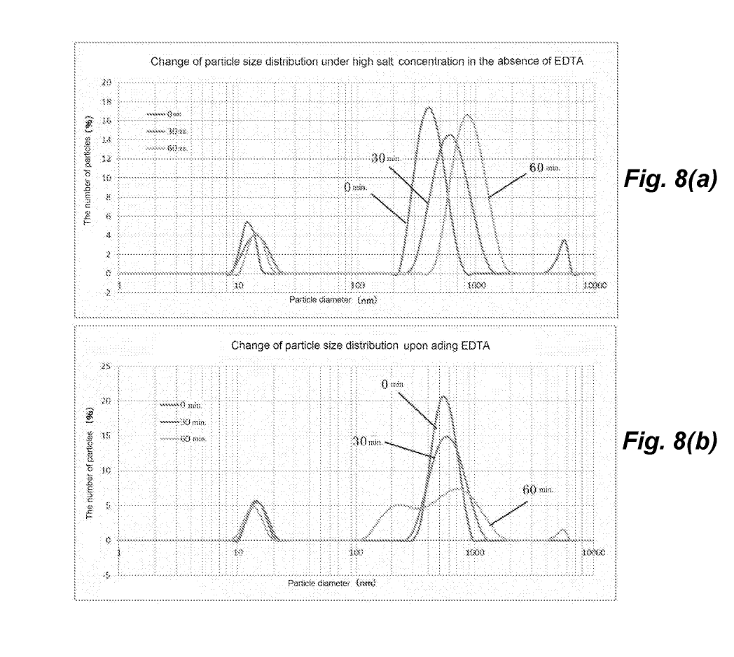

The desalting agent that is effective in the present invention can be selected as described below. Specifically, judgment can be made by evaluating a difference of behavior of silica particles in physiological saline depending on presence or absence of addition of the desalting agent. The desalting agent can be selected by the behavior in which an average particle diameter increases with time under non-existence of the desalting agent, and the average particle diameter is maintained in the existence of the desalting agent. That is, when no desalting agent is added, electric charge on a particle surface is counteracted by the salt, and reduction of dispersibility of the particles starts to cause aggregation of the particles, resulting in an increased apparent particle diameter in dynamic light scattering (DLS) measurement. In contrast, addition of the desalting agent causes trapping of salt components (sodium ions or the like), and the electric charge on the particle surface is maintained. Thus, aggregation of the particles with each other can be prevented. A specific example is shown in FIG. 8.

In the present invention, it is also preferable to use an aptamer as the desalting agent. The aptamer means a nucleic acid molecule, peptide or the like that specifically binds with a specific molecule or atom. In the present invention, it is preferable to use an aptamer that specifically binds with the salt component (alkali component or acid component) and is effective in removing an alkali from the inside of the system. As the aptamer having such properties, those described in the paper shown below can be used, for example. In this paper, an aptamer that has affinity with potassium ions is disclosed.

“Colorimetric detection of potassium ions using aptamer-functionalized gold nanoparticles” Zhengbo Chena, Yanqin Huangb, Xiaoxiao Lia, Tong Zhoua, He Maa, Hong Qianga, Yifei Liva,

### a Department of Chemistry, Capital Normal University, Beijing 100048, China

### b College of Chemistry and Chemical Engineering, Xinxiang University, Xinxiang 453003, China

### [Developing Fluid]

The developing fluid related to a preferred embodiment of the present invention contains the target substance and the abode-described desalting agent. On this occasion, the developing fluid is preferably one containing the salt component (alkali component or acid component), and further preferably one that is neutralized with an acid or alkali after heating. The heating is performed preferably at a high temperature, and particularly preferably at a boiling temperature (a little higher than 100° C.). After being neutralized with the acid, the developing fluid is preferably sufficiently mixed using a vortex mixer or the like.

In the conventional developing fluid, the particles aggregate in the presence of the salt, and aggregates exceeding membrane hole sizes increase. As a result, the silica particles cannot arrive at test lines. In contrast, according to the present invention, aggregation of particles (particularly the silica particles) can be prevented by adding the specific desalting agent. Therefore, the particles that can pass through the membrane hole sizes increase, and the particles can be efficiently carried to the test lines. As a result, such fluid can lead to sensitivity improvement.

### [Meaning of Technical Expressions]

For clarification of the technical expressions used in this specification, the target substance 1 (although the reference numerals and symbols are denoted in FIG. 1, the present invention is not construed as being limited thereto) is a substance as an object for detection by lateral flowmetry, and it has the same meaning as the test substance in an analyte. Each of the binding substances 2b and 3b indicates a substance having binding property to the target substance and the capturing substance, respectively, and it is preferably a biomolecule. The labeling particles 2a and 3a introduced with a labeling substance are referred to as the labeling units 2 and 3. However, in broad sense, the term “labeling particles” may be also used to have a meaning including a labeling unit. Meanwhile, that immobilized to the membrane in the test area and capturing the labeling unit 2 by way of the target substance 1 is the test purpose capturing substance 4. Meanwhile, that immobilized to the membrane in the reference area is the reference purpose capturing substance 5, and the labeling unit 3 is bound thereto without being mediated by the target substance 1.

Further, as described herein, the substance not only means a chemical compound and a chemically synthesized molecule but also includes a biological molecule (e.g., protein, peptide, and nucleic acid), and it may have either an artificial origin or a natural origin. In broad sense, it includes cells of a living body, microorganisms (bacteria or the like), and viruses. In addition, the binding or connecting generally indicates a state in which plural pieces are assembled into a continuous and integral form, and, in addition to a chemical bond such as a covalent bond, an ionic bond, and a hydrogen bond, it includes chemical adsorption, physical adsorption, other physical link states such as assembling, a screw bond, and interlocking, or the like. As described herein, the binding may be a direct binding between plural pieces or an indirect binding via other piece.

Analyte

The analyte S used for the present embodiment is not specifically limited, and examples thereof include a clinical analyte represented by body fluid or feces of a human or an animal such as blood, plasma, serum, lymph fluid, urine, saliva, pancreatic fluid, stomach fluid, sputum, and swab collected from mucous membrane of a nose or a neck; a food analyte represented by liquid food, semi-solid food, and solid food; an analyte sampled from nature such as soil, river, and sea water; an analyte obtained by wiping of production line or a clean room in a plant; and an analyte sample from an environment represented by a sampling analyte by air sampler. When it is a liquid state, the analyte may be used as it is. When it is a semi-solid or solid state, it may be used after undergoing a treatment such as dilution and extraction.

Binding Substance

In the present embodiment, the test purpose binding substance 2b is used, being integrated with the labeling particle 2a (labeling unit 2). Specific examples of the binding substance 2b include, although not specifically limited, a biological molecule having a binding property to the target particles, specifically, an antibody. The binding substance may be directly bound and integrated with the target particles, or may be indirectly bound with the target particles via other substance. Binding between the target particles and the binding substance may be performed by a common method such as a physical adsorption based on hydrophobic interaction and a chemical binding method with an aid of a functional group like a binding between a succinimido group and an amino group or a binding between a maleimide group and a thiol group. When the target particles are fine particles, plural binding substances may bind to the surface of one labeling unit. Further, as for an embodiment of a labeling unit in which fluorescent silica fine particles as labeling particles are integrated with a binding substance, reference can be made to WO 2008/018566.

According to the present embodiment, the reference purpose binding substance 3b is also used separately from the test purpose binding substance 2b. The reference purpose binding substance 3b has a binding property to a reference purpose capturing substance 5 which is described below. In addition, the labeling particles 3a and the reference purpose binding substance 3b are integrated to form the reference purpose labeling unit 3. Thus, during the course of migration along the membrane, the labeling unit 3 is captured by the capturing substance 5. A binding and integration mode between the labeling particle 3a and the binding substance 3b or the preferred material type of the binding substance 3b is the same as those of the test purpose binding substance 2b. However, the reference purpose binding substance 3b preferably has no binding property to the test purpose capturing substance 4 and the target substance. Meanwhile, the test purpose binding substance 2b may have a binding property to the reference purpose capturing substance 5, but more preferably has no binding property thereto.

In addition, the labeling particles may be applied in one kind, or in the form of using only the test purpose and the reference purpose 2a (2b) without using 3a (3b).

Test Purpose Capturing Substance

In the membrane used for the present embodiment, the test purpose capturing substance 4 is immobilized onto the membrane material. The capturing substance 4 has a binding property to the target substance 1 in order to capture a complex including the labeling particle 2a, the binding substance 2b, and the target substance 1. Since the capturing substance 4 has this binding property, capturing of a complex consisting of the labeling unit 2 and the target substance 1 can be achieved. As a result, a fluorescence-emitting line or a colored line by light absorption, caused by the labeling unit 2, is formed in the test area nₜ. Examples of a combination of the “biding substance”-“target substance”-“capturing substance” include the following, but the present invention is not limited to them: antibody (B)-antigen (C) against antibody (B)-antibody (D) against antigen (C), antigen (E)-antibody (F) against antigen (E)-antibody (G) against antibody (F), nucleic acid (H)-nucleic acid (I) with a sequence complementary to nucleic acid (H)-nucleic acid (J) with a sequence complementary to nucleic acid (I) but different from sequence of nucleic acid (H), receptor (K)-ligand (L) for receptor (K)-antibody (M) against ligand (L), aptamer (N)-protein (O) specifically binding to aptamer (N)-aptamer (P) specifically binding to protein (O) at a site different from aptamer (N), aptamer (Q)-protein (R) specifically binding to aptamer (Q)-antibody (S) against protein (R).

Reference Purpose Capturing Substance

In the present embodiment, the reference purpose capturing substance 5 is immobilized in the reference area nᵣ of the membrane, and it directly binds to the reference purpose binding substance 3b without being mediated by the target substance 1. Thus, when the labeling unit 3 mixed in the flowing analyte liquid s migrates without being bound to the target substance 1, it can directly capture the labeling unit 3 (see FIG. 2). As a result, a line with light absorption or fluorescence from the labeling unit 3 is formed in the reference area nᵣ. The reference purpose capturing substance 5 is not specifically limited, and examples thereof include a biological molecule having a binding property to a binding substance. Specific examples thereof include an antibody.

The reference purpose capturing substance 5 in the reference area and the test purpose binding substance 2b may have a binding property. For such case, the test purpose labeling particle 2a is captured in the reference area nᵣ. Specifically, both the labeling particles 3a and the labeling particles 2a are captured in the reference area, and even in such case, colored or fluorescent state of the labeling particles in the reference area can be visually recognized. Meanwhile, since the fluorescent labeling unit 2 having the target substance is already captured in the test area, there would be no problem for general use.

Materials

Materials of each constitutional member which may be used for the planar test piece (test strip) 10 of the present embodiment is not specifically limited, and any common member used for immunochromatographic test strip can be used. Preferred examples of the sample pad and the conjugate pad include a pad of glass fiber such as Glass Fiber Conjugate Pad (trade name, manufactured by MILLIPORE). Preferred examples of the membrane include a nitrocellulose membrane such as Hi-Flow Plus120 (trade name, manufactured by MILLIPORE). Preferred examples of the absorption pad include a cellulose membrane such as Cellulose Fiber Sample Pad (trade name, manufactured by MILLIPORE). When the adhesive-attached backing sheet is used, examples thereof include AR9020 (trade name, manufactured by Adhesives Research).

Introduction of Labeling Particles to Conjugate Pad

With regard to the immunochromatography of the present embodiment, it is preferable that coloration particles or fluorescent particles bound with a binding substance, be introduced in advance as a labeling unit to the conjugate pad. The content of labeling particles per unit area (cm²) of the conjugate pad is, although not specifically limited, preferably 20 μg/cm² to 2 mg/cm² and more preferably 20 to 200 μg/cm². When the content thereof is too high, the analyte binding number per single particle is lowered and the detection sensitivity is impaired. As for the introduction method, a method of coating, applying or spraying dispersion of the labeling particles, and drying can be mentioned. At that time, it is also possible that coloration particles or fluorescent particles are contained in advance, dried first, and then introduced with fluorescent particles or coloration particles. Alternatively, the coloration particles and fluorescent particles are mixed with each other in advance and then they can be introduced as mixed colloid.

### [Labeling Agent]

As the labeling agent, those to be applied to these kinds of tests can be appropriately used. However, for example, those in combination of the labeling particles 2a, 3a such as fluorescent or light absorbing silica particles, fluorescent or light absorbing latex particles, semiconductor fine particles or gold colloid particles, with binding biomolecules 2b, 3b can be used. Moreover, the labeling agent needs not to be in a particle form, and may be the biomolecule such as the protein and the antibody, or a composite thereof. In the present invention, a problem of aggregation caused by the above-mentioned salt component (acid component or alkali component) becomes significant. Thus, the present invention preferably corresponds to the embodiment in which the silica particles are particularly used as the labeling agent.

Fluorescent Silica Particles

Method for producing the fluorescent silica particles is not particularly limited, and silica particles obtained by any arbitrary production method can be used. Examples of the method include a sol-gel method described in Journal of Colloid and Interface Science, vol. 159, p. 150-157 (1993).

In the present invention, it is particularly preferable to use silica particles containing functional compounds, which are obtained by a method of producing colloid silica particles containing fluorescent dye compounds as described in WO 2007/074722. Specific examples of the functional compounds include a fluorescent dye compound, a light absorbing compound, a magnetic compound, a radioactive-labeled compound, and a pH sensitive dye compound.

Specifically, the silica particles containing functional compounds can be prepared by reacting the functional compounds with a silane coupling agent and performing polycondensation of a product obtained through a covalent bond, ionic bond, or other chemical bonds or by adsorption with one or more of silane compounds to form a siloxane bond. Accordingly, the silica particles consisting of the organosiloxane component and the siloxane component that are bound to each other via siloxane bond are obtained.

As a preferred mode of producing the silica particles containing the functional compounds, production can be made by reacting the functional compounds having or provided with an active group such as an N-hydroxysuccinimide (NHS) ester group, a maleimide group, an isocyanate group, an isothiocyanate group, an aldehyde group, a para-nitrophenyl group, a diethoxy methyl group, an epoxy group, and a cyano group with a silane coupling agent having a substituent which reacts with those active groups (e.g., an amino group, a hydroxy group, and a thiol group), and condensing and polymerizing the product obtained by forming a siloxane bond after forming a covalent bond with one or more types of silane compounds.

The following example relates to a case in which APS and tetraethoxy silane (TEOS) are used as a silane coupling agent and a silane compound, respectively.

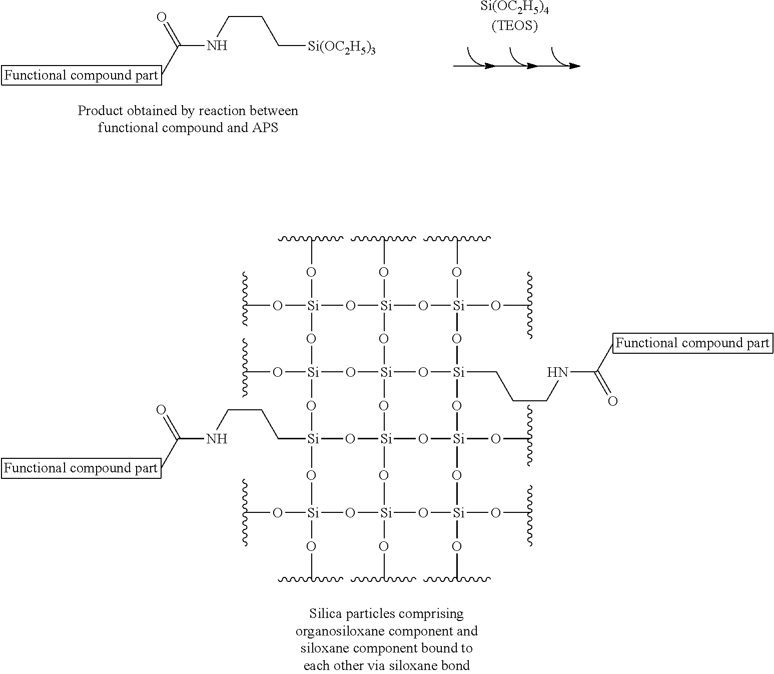

Specific examples of the functional compound having or provided with an active group may include NHS ester group-containing fluorescence dye substances such as 5- (and -6)-carboxytetramethylrhodamine-NHS ester (trade name, manufactured by emp Biotech GmbH), DY550-NHS ester or DY630-NHS ester represented as follows (each trade name, manufactured by Dyomics GmbH).

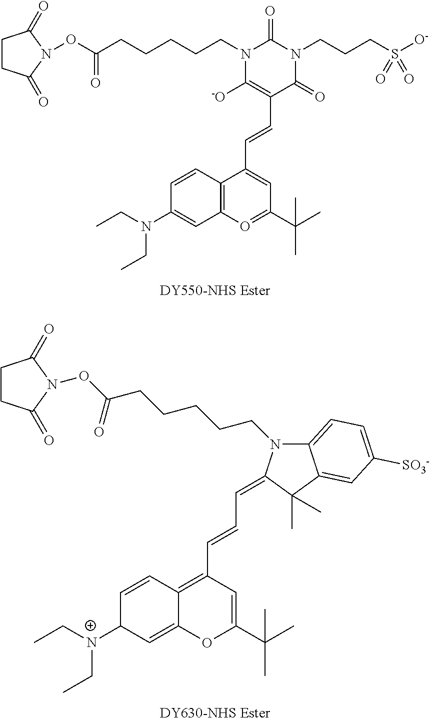

Examples of the substituent-containing silane-coupling agent include an amino group-containing silane-coupling agent such as γ-aminopropyltriethoxysilane (APS), 3-[2-(2-aminoethylamino)ethylamino]-propyltriethoxysilane, N-2-(aminoethyl)-3-aminopropylmethyldimethoxysilane, and 3-aminopropyltrimethoxysilane. Among them, APS is preferable.

The silane compound to be condensed and polymerized is not particularly limited, and examples thereof include TEOS, γ-mercaptopropyltrimethoxysilane (MPS), γ-mercaptopropyltriethoxysilane, γ-aminopropyltriethoxysilane (APS), 3-thiocyanatopropyltriethoxysilane, 3-glycidyloxypropyltriethoxysilane, 3-isocyanatopropyltriethoxysilane, and 3-[2-(2-aminoethylamino)ethylamino]propyl-triethoxysilane. Among them, TEOS is preferable from the point of view of forming the siloxane component to be contained in the silica particles, and besides MPS and APS are preferable from the point of view of forming the organosiloxane component to be contained in the silica particles.

According to the production as described above, spherical or almost spherical silica particles can be prepared. Herein, “almost spherical particles” mean particles having a (major axis)/(minor axis) ratio of 2 or less.

For obtaining silica particles having a desirable average particle diameter, it is possible to remove particles having an excessively large particle diameter or an excessively small particle diameter by ultrafiltration by using an ultrafiltration membrane such as YM-10 or YM-100 (each trade name, manufactured by Millipore Corporation) or by recovering only a supernatant or precipitates after performing centrifugal separation with suitable acceleration of gravity.

As a biomolecule (binding substance) to be combined or absorbed on the surface of the silica particles, there includes antigens, antibodies, DNAs, RNAs, sugars, polysaccharides, ligands, receptors, proteins, peptides and the like. Here, the term “ligand” means a substance capable of specifically binding to a protein, and examples thereof include substrates capable of binding to enzyme, coenzymes, regulatory factors, hormones, neurotransmitters, and the like. Thus, the ligands include low-molecular weight molecules or ions as well as high-molecular weight substances.

The average particle diameter of the labeling particles (preferably fluorescent silica particles) is preferably from 1 nm to 1 μm, more preferably from 20 nm to 500 nm, and more preferably from 50 nm to 300 nm.

In the present invention, the average particle diameter is an average diameter of the circle (average circle-equivalent diameter) obtained by measuring the total projected area of 100 pieces of randomly-selected labeling reagent silica particles for example in an image obtained under transmission electron microscope (TEM) or scanning electron microscope (SEM) using an image processing equipment, dividing the total area with the number of the labeling reagent silica particles (100 pieces), and determining the circle having an area equivalent to that.

Further, the “average particle diameter” indicates an average particle diameter of particles consisting of only primary particles, which is different from the “particle size according to a dynamic light scattering method” described below having a concept including secondary particles formed by aggregation of primary particles.

As described herein, the “particle size according to the dynamic light scattering method” is measured by the dynamic light scattering method, and it is a concept including secondary particles formed by aggregation of primary particles as well as primary particles, different from average particle diameter. This particle size is an indicator for evaluating dispersion stability of the complex particles described above.

Examples of a device for measuring the particle size according to the dynamic light scattering method include Zetasizer Nano (trade name, manufactured by Malvern Instruments Ltd.). According to the method, fluctuation in light scattering intensity over time that is caused by light scatterers such as fine particles is measured, the speed of the light scatterers in Brownian motion is calculated based on an autocorrelation function, and the particle size distribution of the light scatterers is determined based on the results.

The fluorescent silica particles preferably have monodispersion as a granular substance. The variation coefficient, so-called CV value, of the particle size distribution is not specifically limited, but preferably 10% or less, and more preferably 8% or less.

Latex Particles

In the present invention, it is preferable to use the above-described silica fine particles because the effect thereof is significant, but in addition thereto or in place thereof, latex particles may be used as the labeling particles. Examples of the latex particles may include synthetic polymer particles consisting of a polystyrene, styrene-sulfonic acid (salt) copolymer, styrene-methacrylic acid copolymer, acrylonitrile-butadiene-sulfonic acid copolymer, vinyl chloride-acrylic acid ester copolymer, or vinyl acetate-acrylic acid ester copolymer. Further, as for the method of coloring the latex particles, methods disclosed in JPA-2000-178309 (“JP-A” means unexamined published Japanese patent application), JP-A-10-48215, JP-A-8-269207, JP-A-6-306108, or the like can be used. Immobilization of a fluorescent substance (labeling substance) for those kinds of particles can be suitably performed according to a common method. For example, reference can be made to JP-T-2005-534907 (“JP-T” means published searched patent publication), JP-A-2010-156642, JP-A-2010-156640, or the like. As an example of commercially available fluorescent latex particles, xMAP (registered trademark) Multi-Analyte COOH Microspheres manufactured by Luminex is known (http://hitachisoft.jp/products/lifescience/lineup/luminex/about/bead.htmlhttp://hitachisoft.jp/products/lifescience/pdf/the_luminex_labmap_system.pdf).

Semi-Conductor Particles or the Like

Semi-conductor particles may be used as the labeling agent. Materials of the semi-conductor particles are not specifically limited, but preferred examples thereof include ZnO, ZnS, ZnSe, ZnTe, CdO, CdS, CdSe, CdTe, HgS, HgSe, HgTe, InP, InAs, GaN, GaP, GaAs, TiO₂, WO₃, PbS, and PbSe. For example, semi-conductor fine particles described in Japanese Patent No. 3897285 or the like may be used. The surface of the semi-conductor fine particles may be modified by substituting an atom such as S, O, Se, Te, P, As, and N present on surface of semi-conductor fine particles with a —SH group of a thiol compound. Examples of the gold particles and the metal fine particles that may be used include colloidal gold particles and colloidal metal particles described in JP-A-2003-26638 or the like. Specific examples of the colloidal metal particle include colloidal metal particles of platinum, copper, iron oxide or the like. Examples of the inorganic crystals include iron oxide (III) (Fe₂O₃), silver oxide (I) (Ag₂O), tin oxide (IV) (SnO₂), titanium oxide (IV) (TiO₂), and indium tin oxide (ITO). The inorganic crystals described in JP-A-2005-76064 may be used, for example.

Light Absorption Coefficient

When the labeling agent contains light absorbing fine particles, the light absorbing fine particles preferably absorb visible light and exhibit a color which is visually recognizable. Further, they are the particles having molar absorption coefficient E of preferably 5×10⁶ M⁻¹ cm⁻¹ or higher, more preferably from 5×10⁷ M⁻¹ cm⁻¹ to 1×10¹⁰ M⁻¹ cm⁻¹.

As described herein, the molar absorption coefficient E can be calculated based on the following Lambert-Beer equation.

A=Log₁₀(I₀/I)=εbp=aₛbp′

{A: Absorbance, I: Intensity of transmitted light, I₀: Intensity of incident light, ε: Molar absorption coefficient (M⁻¹ cm⁻¹), b: Light passage length (cm), p: Concentration of labeling particles (including mixture dispersion of coloration particles and fluorescent fine particles) (M (mol/L)), aₛ: Relative absorption coefficient, and p′: Concentration of labeling particles (including mixture dispersion of coloration particles and fluorescent fine particles) (g/L)}

The concentration p′ (g/L) is a value obtained by recovering only the labeling particles from a constant amount (e.g., 1 mL) of dispersion containing labeling particles and determining the dried mass. Meanwhile, the concentration p (mol/L) is a value obtained by determining the size of the labeling particles from a TEM image, calculating the volume of a single particle, determining mass of a single particle in view of density of the particle (e.g., 2.3 g/cm³ for silica particles), recovering only the labeling particles from a constant amount (e.g., 1 mL) of dispersion containing labeling particles, and determining the mole number in view of the dried mass of the labeling particles. In the present specification, the expression “molar absorption coefficient E of the labeling particles” means molar absorption coefficient E of labeling particles in the dispersion, which is obtained by measuring absorbance of dispersion containing the labeling particles and applying the result to the Lambert-Beer equation described above. The absorbance, absorption spectrum, and E of the labeling particles can be measured from dispersion such as aqueous dispersion, ethanol dispersion, or N,N-dimethyl formamide dispersion by using any light absorption spectrophotometer or plate reader.

An embodiment related to surface modification of the light absorbing fine particles or introduction of the binding substance is similar to that of the above-mentioned fluorescent fine particles.

### [Detection Method]

Immunochromatography is a detection method in which the labeling reagent silica fine particles (labeling units) 2, 3 that migrate based on capillary phenomenon or the like are ordinarily used to accumulate the above-described particles in a determination portion and to perform determination. For example, it is preferably performed by immunochromatography or using a micro flow chip or the like. In this case, the labeling reagent silica fine particles may be preferably used as a labeling unit for lateral flowmetry. In addition, in the method of detecting a target substance of the present invention, it is preferable to detect target substances by using lateral flow type immunochromatography.

With regard to a method for producing the test strip, a sample pad, a conjugate pad, an antibody-immobilized membrane, and an absorption pad are overlaid in that order while both ends of each member are attached to the neighboring member such that they are overlapped with each other within a range of 1 to 5 mm (preferably on a backing sheet) so as to easily cause capillary phenomenon between the respective members.

The fluorescence detection system for the immunochromatography preferably consists of at least (1) a test strip consisting of a sample pad, a member impregnated with labeling reagent silica fine particles or labeling reagent silica fine particles for lateral flowmetry containing a fluorescent substance (conjugate pad), an antibody-immobilized membrane and an absorption pad, and (2) an excitation light source.

According to the fluorescence detection system, from the viewpoint of detecting the fluorescence emitted from the labeling reagent silica fine particles (labeling units) by visual observation or the like, it is preferable that excitation light source emits excitation light with a wavelength of from 200 nm to 400 nm. Examples of the excitation light source include a mercury lamp, a halogen lamp, and a xenon lamp. In the present invention, excitation light illuminated from a laser diode or light emitting diode is particularly preferably used.

Further, the fluorescence detection system is preferably equipped with a filter for selectively transmitting light of specific wavelength from the excitation light source. Further, from the viewpoint of detecting only the fluorescence by visual observation or the like, it is more preferably equipped with a filter which is capable of removing the excitation light and transmitting only the fluorescence.

In particular, the fluorescence detection system preferably contains a photomultiplier tube or CCD detector capable of receiving the fluorescence. Accordingly, fluorescence with visually undeterminable intensity or wavelength can be detected, and further quantification of target substances can be made as its fluorescence intensity can be measured, enabling detection and quantification with high sensitivity.

The wavelength of the excitation light is preferably between 300 nm and 700 nm. The wavelength of the fluorescence is preferably a wavelength that can be recognized with the naked eye, i.e., preferably between 350 nm and 800 nm. Further, from the viewpoint of obtaining high visibility when observed with the naked eye, the wavelength is preferably between 530 nm and 580 nm. For such case, the wavelength of the excitation light is preferably between 500 nm and 550 nm for efficient generation of the fluorescence within the wavelength range described above.

From the viewpoint of easy handling by an unskilled individual and also of POCT (Point of Care Testing), the test strip according to a preferred embodiment of the present invention preferably has a housing (i.e., a casing) with an observation window made of plastic materials or the like which allows the detection line of the test strip to be observed with the naked eye. For example, the housing described in JP-A-2000-356638 can be mentioned.

The above term “POCT” means a test for diagnosing a patient at possibly nearest place. Conventionally, a collected analyte like blood, urine, and tissues of lesion is sent to a central test lab of a hospital or a professional test center to obtain the data, and thus it takes a time (e.g. 1 day or more) to have a confirmed diagnosis. However, according to POCT, a fast yet accurate treatment can be made, based on the test information which is supplied in short time. From such point of view, it enables an urgent test or a test during operation at hospital, and thus it is highly needed at actual medical site, in particular.

### EXAMPLES

The present invention will be described in more detail based on Examples given below, but the invention is not meant to be limited by these.

### (Preparation of Silica Fine Particles)

2.9 mg of 5-(and-6)-carboxytetramethylrhodamine.succinimidyl ester (trade name, manufactured by EMP Biotech GmbH) was dissolved in 1 mL of dimethyl formamide (DMF). Then, 1.3 μL of APS was added thereto and the reaction was carried out for 1 hour at room temperature (24° C.).

600 μL of the resulting reaction liquid was admixed with 140 mL of ethanol, 6.5 mL of tetraethoxysilane (TEOS), 35 mL of distilled water, and 15 mL of 28% by mass ammonia water, and the reaction was allowed to occur at room temperature for 24 hours.

The reaction solution was centrifuged at a gravitational acceleration of 15,000×g for 30 minutes, and the supernatant was removed. 4 mL of distilled water was added to the precipitated silica particles for dispersion, and the dispersion was centrifuged again at a gravitational acceleration of 15,000×g for 20 minutes. Further, this washing operation was repeated twice additionally, for removal of the unreacted TEOS, ammonia and others contained in the silica fine particles dispersion. Thus, 1.71 g of silica nanoparticles having an average particle diameter of 200 nm were obtained (Yield ratio: about 97%).

### Comparative Example 1

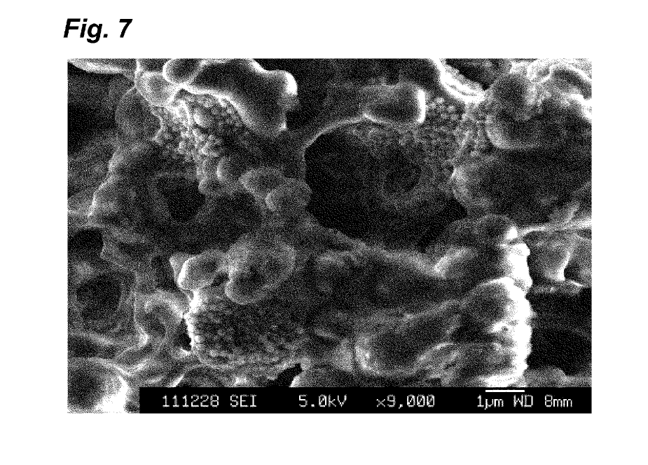

A developing fluid sample was prepared by adding the silica fine particles prepared as described above to a salt component-containing aqueous solution obtained by adding hydrochloric acid in a neutralization amount to 2N sodium hydroxide so as to be contained at 0.02% by mass. A migration test of developing fluid was carried out using this sample. The result is shown in FIG. 5(b). A SEM image on the above occasion is shown in FIG. 7. It is found that the labeling agent (silica fine particles) was locally distributed, and firmly fixed onto surfaces of fibers of the membrane (length: 25 mm, trade name: Hi-Flow Plus 120 Membrane, manufactured by MILLIPORE). FIG. 6 shows the results of FIGS. 5(a) and 5(b) by taking a vertical axis as a ratio of accumulation of the particles.

### Example 1

To the developing fluid in the above-described Comparative Example 1, ethylenediaminetetraacetic acid (EDTA) was added to be 1% by mass. A migration test of developing fluid was carried out by using this sample in a manner similar to Comparative Example 1. FIG. 5(a) shows the result.

As found in the comparison of FIG. 5(a) with FIG. 5(b), the labeling agent stagnated before arriving at the position (near 8,000 μm) corresponding to the test area (nₜ) in Comparative Example 1. In contrast, in the present invention, an analyte substance appropriately arrived at the position of the test area (nₜ) and was captured at this position. These results show that, according to the present invention, aggregation of the labeling agent and local fixing onto the membrane is inhibited, and preferred flowability is achieved.

### Example 2

Similar migration tests of developing fluid were carried out by using EGTA or DTPA in place of EDTA as the above-described desalting agent. As a result, a good aggregation inhibiting effect similar to EDTA was found.

### Reference Example

FIGS. 8(a) and 8(b) show the results of measuring particle size distributions with change over time after adding silica particles to a NaCl salt with a high concentration (6% by mass). A numerical value of each curve in FIGS. 8(a) and 8(b) means time (second) elapsed from starting the test. FIG. 8(a) shows that those having an average particle diameter of about 400 nm at addition grew larger to 600 nm after 30 minutes and 800 nm after 1 hour. In contrast, when EDTA was added, the average particle diameter of 500 nm was at addition, and no significant change was found even with lapse of time (FIG. 8(b)). This result shows that electric charge on surfaces of the particles can be maintained by adding EDTA. This is considered to be caused by no occurrence of aggregation in the particles to result no change in the particle diameter (size) of aggregates of the particles. In addition, the particle diameter was larger at 0 minute in the present invention, but a difference between 400 nm and 500 nm was insignificant, and it is important that no progress aggregation was made even if time elapses.

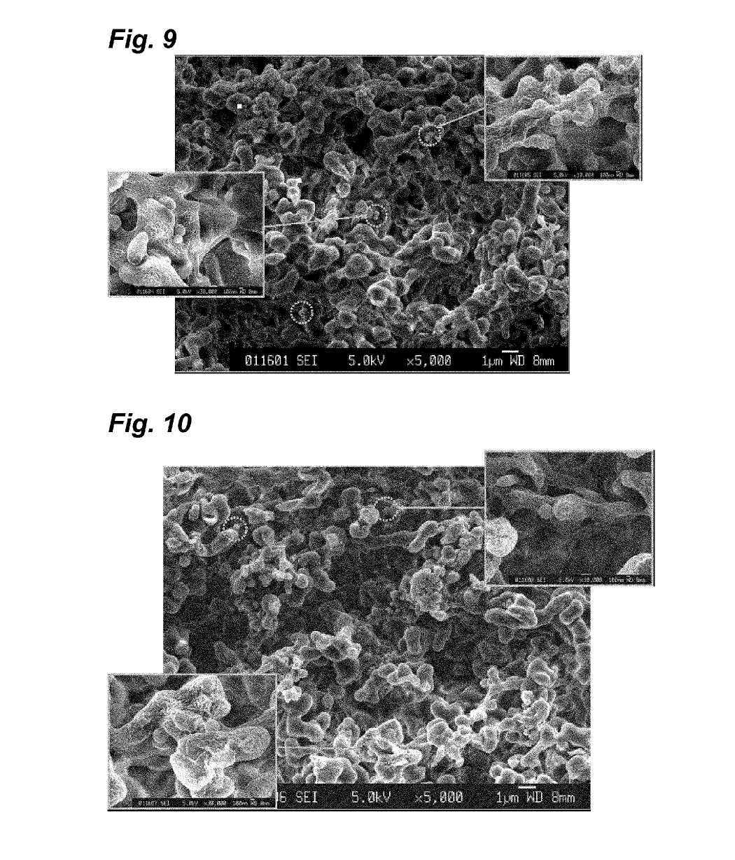

FIG. 9 and FIG. 10 each are a micrograph illustrating a state in which a pad (prepared by immobilizing a desalting agent with a connecting material) subjected to desalting treatment in another Example was used. No large aggregate was found and particles existed in a scattered manner. Six particles per visual field (24 μm per square) were observed. This result also shows that, in Examples according to the present invention, a jam of the labeling particles is significantly improved.

### REFERENCE SIGNS LIST

1 Target substance (analyte substance)
2 Labeling unit
2a Labeling particle
2b Test purpose binding substance
3 Labeling unit
3a Labeling particle
3b Reference purpose binding substance
4 Test purpose capturing substance
5 Reference purpose capturing substance
6 Casing
61 Detection opening part
62 Opening part for introducing analyte
6a Upper casing part
6b Lower casing part
8a Aggregation inhibiting pad
8b Conjugate pad
8c Membrane
8d Absorbent pad
8g Sample pad
9 Desalting agent
10 Test strip
100 Long test body
nᵣ Reference area
nₜ Test area
L Lateral flow direction
S Analyte

Having described our invention as related to the present embodiments, it is our intention that the invention not be limited by any of the details of the description, unless otherwise specified, but rather be construed broadly within its spirit and scope as set out in the accompanying claims.

1. A test piece for immunochromatography, comprising:
an aggregation inhibiting pad;
a conjugate pad; and
a membrane;
wherein the membrane contains a test area for capturing a target substance;
wherein the aggregation inhibiting pad contains a desalting agent; and
wherein the conjugate pad contains a labeling agent.

2. The test piece for immunochromatography according to claim 1, wherein the desalting agent is a chelating agent or an aptamer.

3. The test piece for immunochromatography according to claim 2, wherein the chelating agent is an aminocarboxylic acid-series chelating agent.

4. The test piece for immunochromatography according to claim 1, wherein the labeling agent is fluorescent silica fine particles.

5. The test piece for immunochromatography according to claim 1, wherein a connecting molecule is introduced into the aggregation inhibiting pad, and the desalting agent is introduced thereinto through the connecting molecule.

6. Developing fluid for immunochromatography, comprising:
an alkali or acid;
a target substance; and
a desalting agent.

7. The developing fluid for immunochromatography according to claim 6, wherein the desalting agent is a chelating agent or an aptamer.

8. The developing fluid for immunochromatography according to claim 7, wherein the chelating agent is an aminocarboxylic acid-series chelating agent.

9. The developing fluid for immunochromatography according to claim 6, which is treated through a step of providing analyte liquid with an alkali or acid, a step of heating, and a step of neutralization with the alkali or acid.

10. Immunochromatography, which is performed using the test piece according to claim 1,
wherein the test piece is provided with developing fluid containing an alkali or acid and a target substance, to detect a target substance in the analyte liquid.

11. Immunochromatography, wherein a test piece for immunochromatography provided with a membrane having a test area for capturing a target substance is provided with the developing fluid containing a desalting agent according to claim 6, to detect the target substance.

12. Immunochromatography for performing testing by providing developing fluid containing an alkali or acid and a target substance and allowing passage through a membrane to capture the target substance in a test area of the membrane,
wherein the developing fluid is desalted by using a desalting pad into which a desalting agent is incorporated, or applying desalting treatment to analyte liquid, to prevent aggregation of the target substance.
